(19) United States
(12) Patent Application Publication
(10) Pub. No.: US 2024/0207201 A1
(43) Pub. Date: Jun. 27, 2024

(54) COMPOSITIONS AND METHODS FOR IMPROVING CARDIOVASCULAR HEALTH

(71) Applicant: Maravilla, LLC, Birmingham, MI (US)

(72) Inventors: Nicole Stringham, Phoenix, AZ (US); John Nolan, Waterford (IE); Marina Green Gomez, Waterford (IE)

(21) Appl. No.: 18/394,591

(22) Filed: Dec. 22, 2023

Related U.S. Application Data
(60) Provisional application No. 63/435,423, filed on Dec. 27, 2022.

(51) Int. Cl. A61K 31/047; A61K 31/07; A61K 31/14; A61K 31/197; A61K 31/202; A61K 31/355; A61K 31/375; A61K 31/4188; A61K 31/4415; A61K 31/455; A61K 31/51; A61K 31/519; A61K 31/525; A61K 31/593; A61K 31/714; A61K 33/04; A61K 33/06; A61K 33/18; A61K 33/26; A61K 33/30; A61K 33/32; A61K 33/34; A61K 36/9068; A61P 9/00

(52) U.S. Cl. CPC A61K 31/047; A61K 31/07; A61K 31/14; A61K 31/197; A61K 31/202; A61K 31/355; A61K 31/375; A61K 31/4188; A61K 31/4415; A61K 31/455; A61K 31/51; A61K 31/519; A61K 31/525; A61K 31/593; A61K 31/714; A61K 33/04; A61K 33/06; A61K 33/18; A61K 33/26; A61K 33/30; A61K 33/32; A61K 33/34; A61K 36/9068; A61P 9/00

(57) ABSTRACT

A method for improving cardiovascular health in a human subject includes administering a composition to the human subject identified as being at risk for cardiovascular disease. The composition includes macular xanthophylls. In certain instances, the composition may be provided as a single consumable including lutein, zeaxanthin, and meso-zeaxanthin. The single consumable may include 10 milligrams of lutein, 2 milligrams of zeaxanthin, and 10 milligrams of meso-zeaxanthin. In other instances, the composition may be provided as two or more consumable. The two or more consumable collectively including lutein, zeaxanthin, and meso-zeaxanthin.

### CROSS-REFERENCE TO RELATED APPLICATIONS

This application claims the benefit of U.S. Provisional Application No. 63/435,423 filed on Dec. 27, 2022. The entire disclosure of the above application is incorporated herein by reference.

### FIELD

The present disclosure relates to compositions and methods of preparing and using the same, including methods of using the compositions to improve cardiovascular health, including, for example, managing, reducing, eliminating, and/or mitigating atherosclerotic plaque formation.

### BACKGROUND

This section provides background information related to the present disclosure which is not necessarily prior art.

In the human body, chronic systemic inflammation and oxidation are primary contributors to impaired physiologic function and the development of degenerative diseases, including, for example, atherosclerosis within the cardiovascular system. Indeed, oxidative stress is one of the most salient risk factors in the pathogenesis of atherosclerosis. Mechanistically, the oxidation of low-density lipoprotein (LDL) particles within the vascular endothelium has been hypothesized to be the initial event that occurs in the formation of atherosclerotic plaques. In fact, 40 years ago, it was noted that circulating low-density lipoprotein must be structurally modified before it can contribute to atherogenesis; subsequently, it was discovered that the uptake of low-density lipoprotein by macrophages (to produce foam cells) requires lipid oxidation. Once low-density lipoprotein is oxidized it is recognized by low-density lipoprotein receptors and is phagocytized by macrophages. When this occurs within the arterial wall, the macrophage (now foam cell) cannot leave the tissue and will incorporate itself into the atherosclerotic plaque. Over time, the constant addition of foam cells and oxidized lipids to these plaques causes a hardening of arteries, vessel occlusion, and inflammation. In addition to its contribution to initial macrophage activation and foam cell production, systemic overload of oxidized low-density lipoprotein (OxLDL) triggers a cascade of inflammatory cytokines, creating a positive feedback loop of plaque formation and cardiovascular stress.

Systemic oxidative stress is the product of an imbalance between reactive oxygen species (ROS)/free-radical production and the antioxidant defense system. There is scientific consensus that, within the cardiovascular system, atherosclerosis is the result of a chronic state of high oxidative stress that manifests as oxidative products (lipids and proteins) within the vascular wall. As noted above, the physiologic response to oxidative products is the activation of the innate immune system-namely the activation of macrophages, which function as physiologic scavengers. In addition to the initial macrophage activation and foam cell production, systemic overload of oxidized low-density lipoprotein triggers a cascade of inflammatory cytokines (e.g., interleukin 1 beta; IL-1β) that serves to recruit more white blood cells (e.g., monocytes (i.e., macrophage precursor cells) and T-lymphocytes), which secrete even more inflammatory cytokines (i.e., tumor necrosis factor alpha; TNF-α), and therefore, create a positive feedback loop of plaque formation and cardiovascular stress (as illustrated in FIG. 1A).

Ultimately, for low-density lipoprotein to contribute to plaque formation it must cross the vessel endothelium. Regulation of low-density lipoprotein transcytosis across the endothelium is initiated by the binding of low-density lipoprotein to scavenger receptor B1 (SR-B1) and activin A receptor-like type 1 and is upregulated by the presence of inflammatory cytokines TNF-α, serum amyloid A, IL-1β, and C-reactive protein. Moreover, reactive oxygen species within the circulation facilitate the breakdown of the protective endothelial surface layer (i.e., glycocalyx), a process that is enhanced by presence of oxidized low-density lipoprotein, resulting in an even thinner or disrupted layer of glycocalyx in the presence of oxidized low-density lipoprotein. The presence of reactive oxygen species also increases the permeability of the endothelium via calveolae-mediated transcytosis of low-density lipoprotein. Furthermore, when oxidized low-density lipoprotein is taken up by endothelial cells it activates surface adhesion molecules that promote the adhesion and migration of monocytes from circulation into the subendothelial layers and their subsequent activation/transformation into macrophages. Oxidized low-density lipoprotein is a trigger for leukocyte adhesion to endothelial cells and forms the foundation of many of the hypotheses underlying the process of cardiovascular plaque formation. The oxidative-modification and inflammatory hypotheses illustrate the role that oxidative stress and inflammatory feedback plays in the formation of endothelial plaques and highlights points in the cascade where an antioxidant might act to attenuate the process of inflammation and oxidation.

It would be desirable to provide compositions, and method of using compositions, that can help to reduce, eliminate, and/or mitigate inflammation and oxidation and the effects thereof.

### SUMMARY

This section provides a general summary of the disclosure and is not a comprehensive disclosure of its full scope or all of its features.

In various aspects, the present disclosure provides a method for improving cardiovascular health in a human subject.

In at least one example embodiment, the method includes administering a composition to the human subject identified as being at risk for cardiovascular disease (including, for example, those having high low-density lipoproteins cholesterol and/or history of smoking). The composition includes macular xanthophylls.

In at least one example embodiment, the composition may be provided as a single consumable including lutein, zeaxanthin, and meso-zeaxanthin.

In at least one example embodiment, the single consumable may include 10 milligrams of lutein, 2 milligrams of zeaxanthin, and 10 milligrams of meso-zeaxanthin.

In at least one example embodiment, the macular xanthophyll may include lutein, zeaxanthin, and meso-zeaxanthin.

In at least one example embodiment, the composition may be provided as two or more consumables.

In at least one example embodiment, the composition may be administered to the human subject at least three times a week.

In at least one example embodiment, the composition may be administered to the human subject at least two times a week.

In at least one example embodiment, the composition may be administered to the human subject daily.

In at least one example embodiment, the composition may further include an omega-3 fatty acid.

In at least one example embodiment, the omega-3 fatty acid may be provided as a free acid, a salt, a triglyceride, or any combination thereof.

In at least one example embodiment, the omega-3 fatty acid may include docosahexaenoic acid (DHA), eicosapentaenoic acid (EPA), or a combination of docosahexaenoic acid (DHA) and eicosapentaenoic acid (EPA).

In at least one example embodiment, the composition may further include Vitamin A, Vitamin C, Vitamin D3, Vitamin E, Vitamin B1, Vitamin B2, niacin, Vitamin B6, folic acid, Vitamin B12, biotin, panthothenic acid, calcium, iron, iodine, magnesium, zinc, copper, choline, selenium, manganese, ginger, or any combination thereof.

In various aspects, the present disclosure provides another method for improving cardiovascular health in a human subject.

In at least one example embodiment, the method may include administering a composition to the human subject identified as being at risk for cardiovascular disease. The composition may include macular xanthophylls and an omega-3 fatty acid, where the macular xanthophylls includes lutein, zeaxanthin, and meso-zeaxanthin.

In at least one example embodiment, the composition may be provided as a single consumable.

In at least one example embodiment, the single consumable may include 10 milligrams of lutein, 2 milligrams of zeaxanthin, and 10 milligrams of meso-zeaxanthin.

In at least one example embodiment, the composition may be provided as two or more consumables.

In at least one example embodiment, the omega-3 fatty acid may include docosahexaenoic acid (DHA), eicosapentaenoic acid (EPA), or a combination of docosahexaenoic acid (DHA) and eicosapentaenoic acid (EPA).

In at least one example embodiment, the composition may further include Vitamin A, Vitamin C, Vitamin D3, Vitamin E, Vitamin B1, Vitamin B2, niacin, Vitamin B6, folic acid, Vitamin B12, biotin, panthothenic acid, calcium, iron, iodine, magnesium, zinc, copper, choline, selenium, manganese, ginger, or any combination thereof.

In various aspects, the present disclosure may provide a composition for improving cardiovascular health in a human subject.

In at least one example embodiment, the composition includes lutein, zeaxanthin, meso-zeaxanthin macular xanthophylls, and an omega-3 fatty acid.

In at least one example embodiment, the omega-3 fatty acid may include docosahexaenoic acid (DHA), eicosapentaenoic acid (EPA), or a combination of docosahexaenoic acid (DHA) and eicosapentaenoic acid (EPA).

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIGS. 1A and 1B are schematic representations of the infiltration of low-density lipoprotein from the blood into the arterial wall and targets for antioxidants in the process of plaque formation. FIG. 1A illustrates an atherosclerotic vessel with an oxidative environment that highlights the breakdown of glycocalyx by between reactive oxygen species, transport of low-density lipoprotein across the endothelium via calveolae, oxidation of low-density lipoprotein, phagocytosis by macrophages, foam cell culture, release of pro-inflammatory cytokines, incorporation of oxidized lipids and foam cells into atherosclerotic plaque. FIG. 1B illustrates a cardiovascular environment in the presence of macular xanthophylls (including, for example, lutein (L), zeaxanthin (Z), meso-zeaxanthin (MZ)) in accordance with various aspects of the present disclosure, where the macular xanthophylls interact with reactive oxygen species and subsequently decrease oxidation, producing effects that may include, for example, restored glycocalyx, fewer oxidized lipids, decreased foam cell production, and/or fewer pro-inflammatory cytokines that are able to positively feedback and cause inflammation. Although cardiovascular health is discussed here throughout, it should be appreciated that because blood (and its oxidative products or lack thereof) permeate all bodily systems, there are likely positive affects on the nervous system (including, for example, cognitive function) and/or other physiologic systems and/or bodily functions (including, for example, renal, muscular, pulmonary, hepatic, or any combination thereof).

FIGS. 2A-2C are illustrations showing the structures of meso-zeaxanthin in accordance with various aspects of the present disclosure. FIG. 2A shows an ester form of meso-zeaxanthin. FIG. 2B shows meso-zeaxanthin in free form. FIG. 2C shows meso-zeaxanthin diacetate.

FIGS. 3A-3C are illustrations showing the structure of zeaxanthin in accordance with various aspects of the present disclosure. FIG. 3A shows an ester form of (3R,3′R)-zeaxanthin. FIG. 3B shows (3R,3′R)-zeaxanthin in free form. FIG. 3C shows (3R,3′R)-zeaxanthin diacetate.

FIG. 4A-4C are illustrations showing the structure of lutein in accordance with various aspects of the present disclosure. FIG. 4A shows an ester form of (3R,3′R,6R)-lutein. FIG. 4B shows (3R,3′R,6R)-lutein in free form. FIG. 4C shows (3R,3′R,6R)-lutein diacetate.

FIG. 5 is a graphical demonstration illustrating a change in serum total xanthophyll carotenoid concentration after six months of intervention for active and placebo groups, where the measure is a composite of measure serum for lutein, zeaxanthin, and meso-zeaxanthin and breakdown by carotenoid for the active group is L: +0.31 (0.28), Z: +0.02 (0.03), and MZ: +0.08 (0.09) and breakdown by carotenoid for the placebo group is L: +0.02 (0.1), Z: 0 (0.01), and MZ: 0(0).

FIGS. 6A and 6B are graphical demonstrations illustrating change in serum pro-inflammatory cytokines after six months of intervention of active and placebo groups. FIG. 6A is a graphical demonstration illustrating a change in serum TNF-α, pg/mL, p=0.003. FIG. 6B is a graphical demonstration illustrating a change in serum IL-1β, pg/mL, p<0.001, where outliers are indicated by (o).

FIG. 7 is a graphical demonstration illustrating change in serum oxidized low-density lipoprotein after six months of intervention for active and placebo groups, where a mean oxidized low-density lipoprotein decrease in the active group was 90.44 ng/ml (±173.96) and a mean increase in the placebo group was 50.37 ng/ml (±159.55), which is a statistically significant difference between groups (p=0.009), and where outliers are indicated by (o).

FIG. 8 is a graphical demonstration illustrating improvement as related to a starting point of oxidized low-density lipoprotein, where the highest starting point of oxidized low-density lipoprotein is shown to experience bigger improvements as compared to lower starting points of oxidized low-density lipoprotein.

Corresponding reference numerals indicate corresponding parts throughout the several views of the drawings.

### DETAILED DESCRIPTION

Example embodiments are provided so that this disclosure will be thorough and will fully convey the scope to those who are skilled in the art. Numerous specific details are set forth such as examples of specific compositions, components, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps described herein are not to be construed as necessarily requiring their performance in the particular order discussed or illustrated, unless specifically identified as an order of performance. It is also to be understood that additional or alternative steps may be employed, unless otherwise indicated.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

Dietary compositions or supplements (commonly in the form of tablets, capsules, gels, liquids, and/or powders) are often consumed by human subjects to supplemental and/or replace important vitamins and nutrients that are not otherwise sufficiently absorbed via consumed foods and/or to mitigate disease risk and symptoms thereof. For example, in various aspects, dietary compositions or supplements may have the effect of reducing or limiting inflammation and oxidation and thus the occurrence of diseases influenced by the same.

In accordance with various aspects of the present disclosure, macular xanthophylls (including, for example, meso-zeaxanthin and/or zeaxanthin and/or lutein) are prescribed and/or administered to subjects to prevent and/or treat and/or mitigate the risks of various diseases to which chronic systemic inflammation and oxidation are attributable. For example, in at least one example embodiment, macular xanthophylls (including, for example, meso-zeaxanthin and/or zeaxanthin and/or lutein) are prescribed and/or administered to subjects to prevent and/or treat and/or to mitigate cardiovascular disease.

In accordance with various aspects of the present disclosure, macular xanthophylls (including, for example, meso-zeaxanthin and/or zeaxanthin and/or lutein) are consumed by a subject to aid in the prevention and/or treatment and/or mitigation of various diseases to which chronic systemic inflammation and oxidation are attributable. For example, in at least one example embodiment, macular xanthophylls (including, for example, meso-zeaxanthin and/or zeaxanthin and/or lutein) consumed by a subject to aid in the prevention and/or treatment and/or mitigation of cardiovascular disease

In accordance with various aspects of the present disclosure, the macular xanthophylls may be prescribed/administered/consumed in the form of a carotenoid composition. The carotenoid composition prevents and/or treats and/or mitigates various diseases to which chronic systemic inflammation and oxidation are attributable. For example, in at least one example embodiment, the carotenoid composition aids in the prevention and/or treatment and/or mitigation of cardiovascular disease.

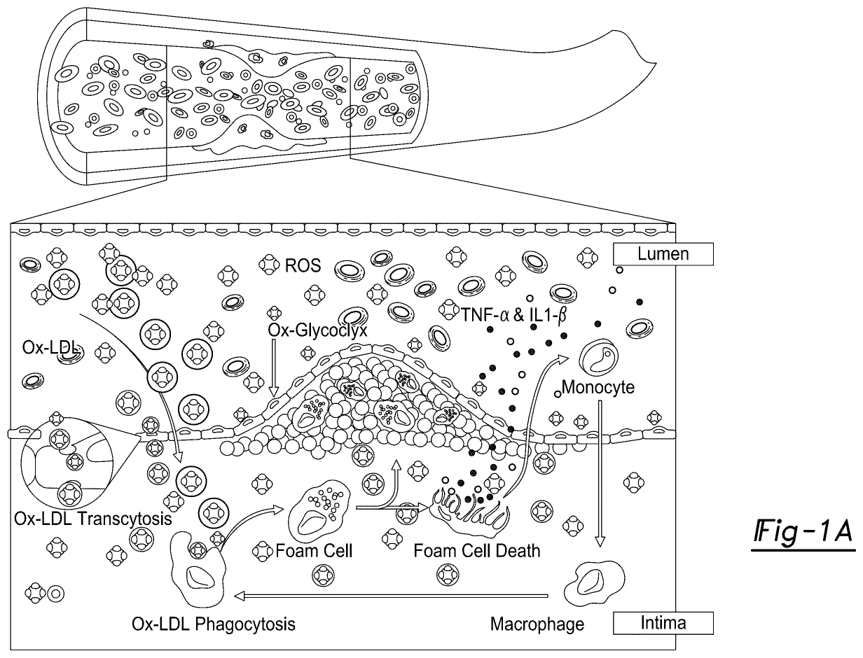

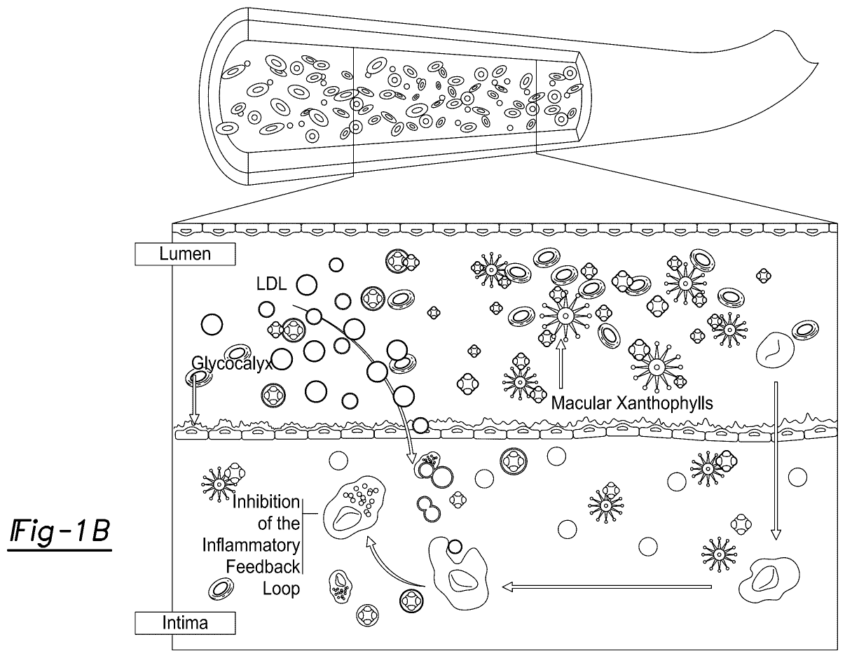

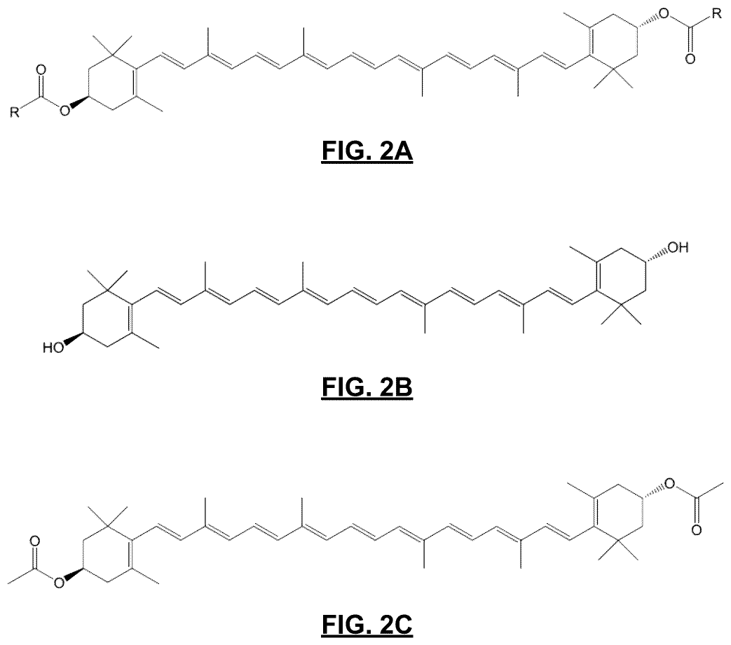

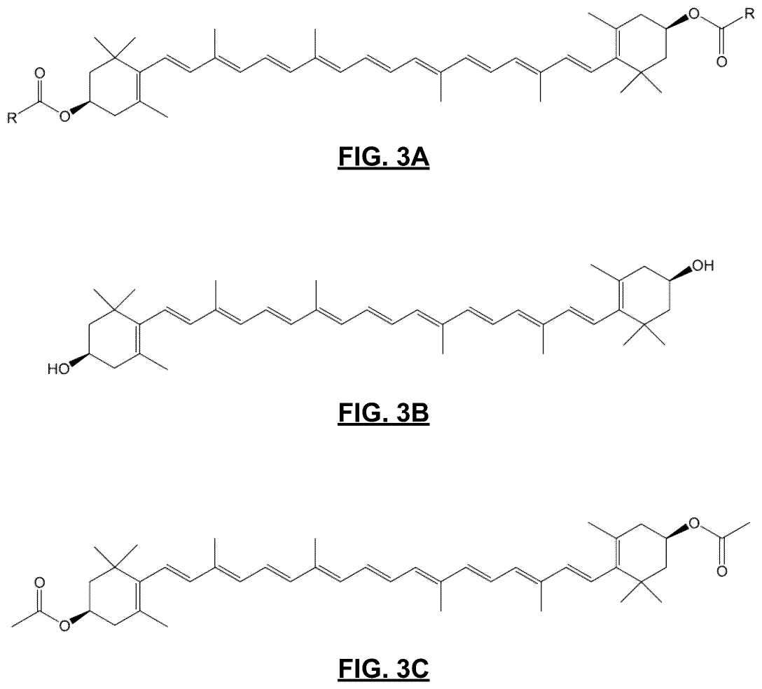

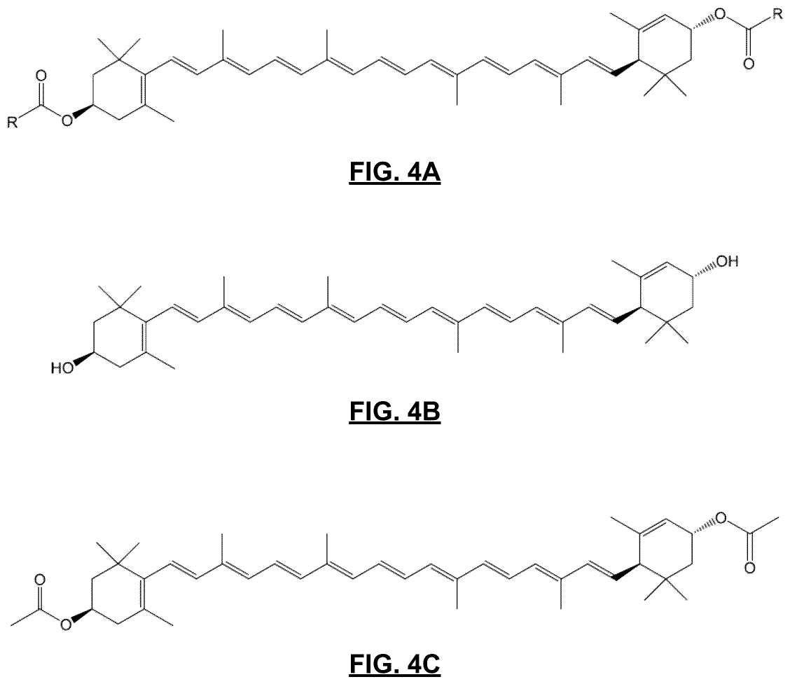

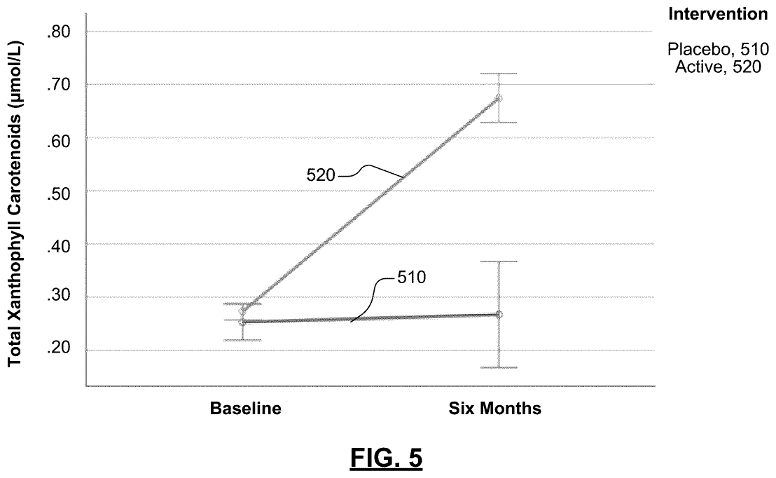

Macular xanthophylls (which may also be referred to as xanthophyll carotenoids) are found in extracts of various plants, including, for example, marigolds. As extracted, the xanthophyll carotenoids are often in the form of esters. FIG. 2A shows meso-zeaxanthin ester, FIG. 3A shows (3R,3′R)-zeaxanthin ester, and FIG. 4A shows (3R,3′R,6R)-lutein ester, where the R is an alkyl chain. Before the prescribing/administering/consuming, the (3R,3′R)-zeaxanthin ester and (3R,3′R,6R)-lutein ester may be subjected to saponification to form their respective structures in free form. FIG. 3B shows (3R,3′R)-zeaxanthin in free form. FIG. 4B shows (3R,3′R,6R)-lutein in free form. The meso-zeaxanthin in free form, as shown in FIG. 2B, may be obtained by a base-catalyzed isomerization of (3R,3′R,6R)-lutein. In at least one example embodiment, the meso-zeaxanthin, (3R,3′R)-zeaxanthin, and (3R,3′R,6R)-lutein in free forms are acetylated to form meso-zeaxanthin diacetate (as shown in FIG. 1C), (3R,3′R)-zeaxanthin diacetate (as shown in FIG. 2C), and (3R,3′R,6R)-lutein diacetate (as shown in FIG. 3C). The carotenoid composition in accordance of the present disclosure may include the meso-zeaxanthin ester, (3R,3′R)-zeaxanthin ester, (3R,3′R,6R)-lutein ester, meso-zeaxanthin in free form, (3R,3′R)-zeaxanthin in free form, (3R,3′R,6R)-lutein in free form, meso-zeaxanthin diacetate, (3R,3′R)-zeaxanthin diacetate, (3R,3′R,6R)-lutein diacetate, or any combination thereof. In each variation, the macular xanthophylls reduces the presence of oxidative low-density lipoproteins (OxLDL), and also, commensurately, IL-1β and/or TNF-α. For example, the macular xanthophylls may act systemically to attenuate oxidative products before those products contribute to atherosclerotic plaque formation.

In at least one example embodiment, the present disclosure provides a carotenoid composition that comprises meso-zeaxanthin ester, (3R,3′R)-zeaxanthin ester, (3R,3′R,6R)-lutein ester, meso-zeaxanthin in free form, (3R,3′R)-zeaxanthin in free form, (3R,3′R,6R)-lutein in free form, meso-zeaxanthin diacetate, (3R,3′R)-zeaxanthin diacetate, (3R,3′R,6R)-lutein diacetate, or any combination thereof. In at least one example embodiment, the present disclosure provides a carotenoid composition that consists essentially of meso-zeaxanthin ester, (3R,3′R)-zeaxanthin ester, (3R,3′R,6R)-lutein ester, meso-zeaxanthin in free form, (3R,3′R)-zeaxanthin in free form, (3R,3′R,6R)-lutein in free form, meso-zeaxanthin diacetate, (3R,3′R)-zeaxanthin diacetate, and (3R,3′R,6R)-lutein diacetate, or any combination thereof. By “consists essentially of,” it is meant that the composition does not intentionally include additional carotenoids, including xanthophyll carotenoids; however, additional carotenoids may be unintentionally included as impurities, such as in concentrations of less than or equal to about 5 wt. %, less than or equal to about 2.5 wt. %, or less than or equal to about 1 wt. % (based on the total weight of the carotenoid composition).

In various aspects, the carotenoid composition in accordance of the present disclosure may include one or more other active ingredients. For example, the carotenoid composition may further include (i.e., in addition to the lutein and/or zeaxanthin and/or meso-zeaxanthin) Vitamin A, Vitamin C, Vitamin D3, Vitamin E, thiamin (also referred to as Vitamin B1), riboflavin (also referred to as Vitamin B2), niacin, Vitamin B6, folic acid (for example, as methylfolate), Vitamin B12, biotin, panthothenic acid, calcium, iron (for example, in fermented bisglycinate form), iodine, magnesium, zinc, copper, choline (for example, as choline bitartrate), omega-3 (e.g., docosahexaenoic acid (DHA), eicosapentaenoic acid (EPA)), selenium, manganese, ginger (e.g., organic ginger), or any combination thereof.

In various aspects, the carotenoid composition in accordance of the present disclosure may include one or more other active ingredients. For example, the carotenoid composition may further include (i.e., in addition to the lutein and/or zeaxanthin and/or meso-zeaxanthin) an omega-3 fatty acid. Unless the context dictates otherwise, the term “fatty acid” is intended to encompass not only the free acid but also derivatives of fatty acids, such derivatives encompassing in particular esters (especially esters formed from glycerol (e.g., monoglycerides, diglycerides and, preferably, triglycerides)) and salts (e.g., containing monocations, such as Na⁺, K⁺ or NH₄⁺). In at least one example embodiment, the omega-3 fatty acid may include an omega-3 polyunsaturated fatty acid or derivative thereof. For example, the omega-3 fatty acid may include docosahexaenoic acid (DHA) or a derivative thereof. In at least one example embodiment, the omega-3 fatty acid may include two or more omega-3 fatty acids. For example, the carotenoid composition may include docosahexaenoic acid (DHA), eicosapentaenoic acid (EPA), or a combination of docosahexaenoic acid (DHA) and eicosapentaenoic acid (EPA). A convenient source of omega-3 fatty acids is fish oil. In at least one example embodiment, the carotenoid composition may include fish oil. Since fish oil has quite a strong odour, it may be preferred to use deodourised fish oil, which is commercially available. Another source of omega-3 fatty acids is nut oil. Other sources of fatty acids may include algae.

In various aspects, the carotenoid composition in accordance of the present disclosure may include one or more other active ingredients. For example, the carotenoid composition may further include (i.e., in addition to the lutein and/or zeaxanthin and/or meso-zeaxanthin) an antioxidant. The antioxidant may include, for example, vitamin E, vitamin C, ascorbyl palmitate, rosemary extract, citric acid, ascorbic acid, tartaric acid, butylated hydroxyanisole (BHA), butylated hydroxytoluene (BHT), potassium sorbate, or any combination thereof. When present, each antioxidant may be individually and independently included at a concentration of greater than or equal to about 20 mg/Kg to less than or equal to about 20 g/Kg (e.g., greater than or equal to about 20 mg/Kg to less than or equal to about 10 g/Kg or greater than or equal to about 10 IU to less than or equal to about 500 IU).

In various aspects, the carotenoid composition in accordance of the present disclosure may include one or more other active ingredients. For example, the carotenoid composition may further include (i.e., in addition to the lutein and/or zeaxanthin and/or meso-zeaxanthin) an alkali metal, an alkaline earth metal, a transition metal or transition metal ion (such as zinc (Zn), zinc ions (e.g., Zn₂⁺), copper (Cu), copper ions (e.g., Cu₂⁺, Cu₃⁺, or combinations thereof)), manganese ions, iron ions, or any combination thereof. The alkali metal can be provided as an ion or salt of lithium (Li, Li⁺), sodium (Na, Na⁺), or potassium (K, K⁺). The alkaline earth metal can be provided as an ion or salt of magnesium (Mg, Mg₂⁺) or calcium (Ca, Ca₂⁺). The transition metal ions can be provided, for example, as transition metal salts, including oxides, sulfates, chlorides, gluconates, stearates, and combinations thereof as non-limiting examples. Exemplary transition metal salts include zinc oxide and cupric oxide, cuprous oxide, iron oxide, and combinations thereof. When present, the alkali metal salt and/or the alkaline earth metal salt and/or the transition metal salt may be present in the composition at individual and independent concentrations of greater than or equal to about 2 mg/Kg to less than or equal to about 200 mg/Kg (e.g., greater than or equal to about 20 mg/Kg to less than or equal to about 100 mg/Kg).

In various aspects, the carotenoid composition in accordance of the present disclosure may include one or more other active ingredients. For example, the carotenoid composition may further include (i.e., in addition to the lutein and/or zeaxanthin and/or meso-zeaxanthin) a carrier at a concentration of greater than or equal to about 50 weight percent to less than or equal to about 90 weight percent (based on the total weight of the carotenoid composition). The carrier may include, for example, a plant extract (such as sunflower oil, soybean oil, canola oil, corn oil, safflower oil, olive oil, citrus coil, or any combination thereof), a vegetable oil, a mineral oil, an animal oil (such as fish oil), glycerine, gelatin (for example, beef and/or pork gelatin), beeswax, fatty acids, or any combination thereof.

In at least one example embodiment, the carotenoid composition in accordance with various aspects of the present disclosure may be provided (e.g., prescribed/administered/consumed) in one or more consumables (e.g., tablets or pills or capsules), where each of the one or more consumables includes nutrients having similarly bioactivity (e.g., lipid v. water solubility). The consumables may each have a mass greater than or equal to about 250 milligrams to less than or equal to about 750 milligrams (e.g., about 250 milligrams, about 300 milligrams, about 350 milligrams, about 400 milligrams, about 450 milligrams, about 500 milligrams, about 550 milligrams, about 600 milligrams, about 650 milligrams, about 700 milligrams, or about 750 milligrams).

In at least one example embodiment, the carotenoid composition may include a total macular xanthophyll concentration less than or equal to about 100 milligrams (e.g., less than or equal to about 75 milligrams or less than or equal to about 50 milligrams or less than or equal to about 20 milligrams). In at least one example embodiment, the carotenoid composition may include a total macular xanthophyll concentration greater than or equal to about 0.5 milligrams (e.g., greater than or equal to about 18 milligrams or greater than or equal to about 20 milligrams or greater than or equal to about 22 milligrams). In at least one example embodiment, the carotenoid composition may include a total macular xanthophyll concentration greater than or equal to about 0.5 milligrams to less than or equal to about 100 milligrams (e.g., greater than or equal to about 0.5 milligrams to less than or equal to about 75 milligrams or greater than or equal to about 0.5 milligrams to less than or equal to about 50 milligrams or greater than or equal to about 0.5 milligrams to less than or equal to about 20 milligrams or greater than or equal to about 20 milligrams to less than or equal to about 75 milligrams or greater than or equal to about 22 milligrams to less than or equal to about 50 milligrams). In at least one example embodiment, the carotenoid composition may include about 10 milligrams meso-zeaxanthin, about 10 milligrams lutein, and about 2 milligrams zeaxanthin.

In various aspects, the carotenoid composition in accordance with various aspects of the present disclosure may be administered at least once a week, at least twice a week, three times a week, or daily. It should be appreciated that the frequency of consumption may be adjusted to account for the concentration of the active agents and also the determined needs of the particular subject.

In various aspects, the present disclosure provides a method to support cardiovascular health in a subject in need thereof. Cardiovascular health as used herein includes, for example, attenuating oxidative products before those products contribute to atherosclerotic plaque formation. The method may include administering to the subject a safe and therapeutically effective amount of the carotenoid composition. As used herein, the term “therapeutically effective amount” means an amount of a compound that, when administered to a subject is sufficient, either alone or in combination with additional therapies, to effect support cardiovascular health, including, preventing and/or treating and/or mitigating cardiovascular health. The “therapeutically effective amount” will vary depending on, for example, the compound, pharmaceutical composition or pharmaceutical dosage form, the condition treated and its severity, and the age and weight of the patient to be treated. In various aspects, a therapeutically effective amount of the composition provides a dose of each included carotenoid of greater than or equal to about 0.5 milligrams to less than or equal to about 25 milligrams.

Certain features of the current technology are further illustrated in the following non-limiting examples.

### Example 1

Example dietary supplements including macular xanthophylls may be prepared and administered to a human subject in accordance with various aspects of the present disclosure. For example, Table 1 compares different baseline (or starting) variables between a first (or active) group receiving the dietary supplement including macular xanthophylls in accordance with various aspects of the present disclosure and a second (or placebo) group.

TABLE 1

| | | | | | signifi- |
|---|---|---|---|---|---|
| | placebo | | active | | cance |
| Parameter | (n = 14) | | (n = 66) | | (p-value) |
| Age (years) | 45.79 | (±9.37) | 44.77 | (±10.63) | 0.742 |
| Sex, | 7 | (50%) | 31 | (47%) | 0.837 |
| Number of | | | | | |
| Females (%) | | | | | |
| Body Mass | 25.18 | (3.50) | 28.18 | (5.84) | 0.069 |
| Index (kg/m²) | | | | | |
| Lutein, Serum | 0.1813 | (0.05686) | 0.1978 | (0.1104) | 0.431 |
| (μmol/l) | | | | | |
| Zeaxanthin, | 0.0711 | (0.0225) | 0.0745 | (0.0302) | 0.688 |
| Serum (μmol/l) | | | | | |
| IL-1β, Serum | 1.86 | (1.429) | 2.84 | (1.586) | 0.036* |
| (pg/ml) | | | | | |
| IL-6, Serum | 3.86 | (2.51) | 5.06 | (3.38) | 0.214 |
| (pg/ml) | | | | | |
| TNG-α, Serum | 5.721 | (3.732) | 7.478 | (3.763) | 0.116 |
| (pg/ml) | | | | | |
| OXLDL, Serum | 1.4779 | (0.6056) | 1.6948 | (0.6675) | 0.273 |
| (ng/ml) | | | | | |

The active group includes sixty-six participants, while the placebo (or second) group includes fourteen participants. All of the participants were between 18 years old and 65 years old. The placebo group received a daily composition (as a soft gel capsule) including only sunflower oil. Each of the active group participants received a daily composition (as a soft gel capsule) including 10 milligrams of lutein, 2 milligrams of zeaxanthin, and 10 milligrams of meso-zeaxanthin. Thirteen (or sixteen percent) of the participants of the sixty-six total participants received the macular xanthophylls as micromicelles. Thirty-eight (or forty-eight percent) of the participants of the sixty-six total participants received the macular xanthophylls suspended in sunflower oil. Twenty-eight (or thirty-five percent) of the participants of the sixty-six total participants received the macular xanthophylls suspended in omega-3.

Demographic, health, and serum data of active and placebo intervention groups were statistically comparable (p>0.05) at baseline (see Table 1) for almost all parameter measurements. Of note, a statistically significant difference was noted between active and placebo group for IL-1β. For clarity, no adjustment was made in subsequent statistical analysis for IL-1β.

Fasting blood samples were collected from the participants at zero months, three months, and six months using standard venipuncture techniques in nine milliliter serum separation tubes. Serum concentrations of lutein, zeaxanthin, and meso-zeaxanthin were determined by high performance liquid chromatography (HPLC). Serum concentrations of inflammatory cytokines (e.g., IL-6, IL-1β, TNF-α) and oxidized low-density lipoprotein were measured via solid-phase sandwich enzyme linked immunosorbent assay (ELISA) in samples obtained at baseline and at six months. R&D systems Human Quantikine ELISA kits were utilized for cytokine measures (IL-6, IL-1β, TNF-α) and data reported in picograms/milliliters (pg/mL). Cell Biolabs OxiSelect Human kits were used to measure serum oxidized low-density lipoprotein and data reported in nanograms/milliliter (ng/mL). All plates were read with a Tecan Sunrise microplate reader (Tecan Austria GmbH, Untersbergstraße, Austria). Procedure did not deviate from the product protocols and all confidence values were less than 10.

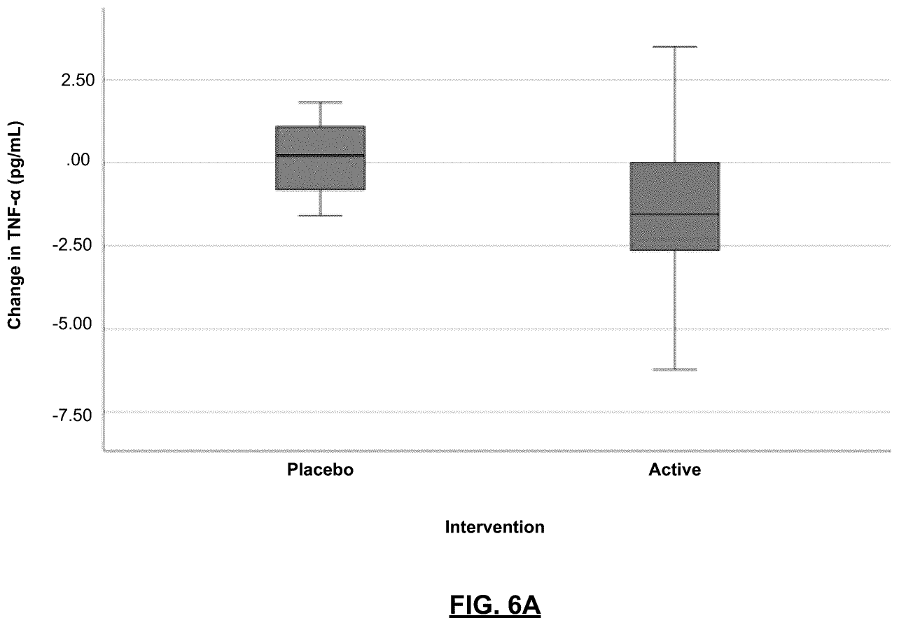

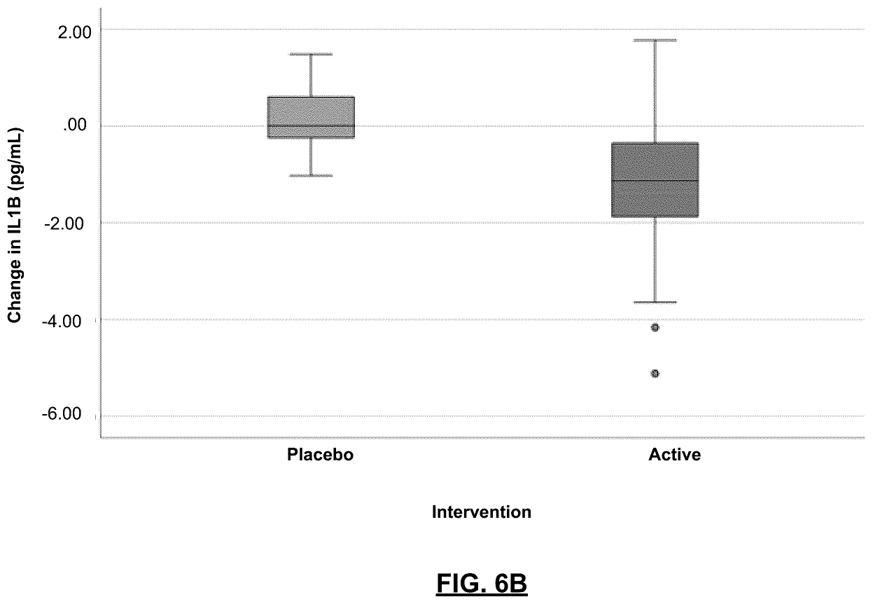

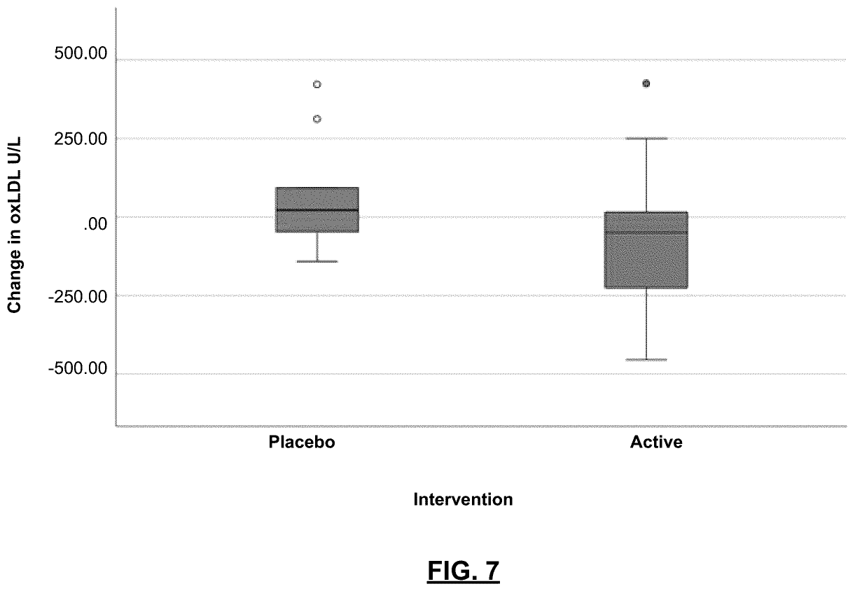

As summarized in Table 1, there were no statistically significant differences between groups for age or body mass index (BMI), as demonstrated by independent samples t-test between placebo and active intervention groups. As illustrated in FIG. 5, analysis of serum via high performance liquid chromatography confirmed an increase in serum concentrations of lutein, zeaxanthin, and meso-zeaxanthin in the combined active group (see reference 520) that was statistically significant compared to placebo (see reference 510) (p<0.001). As illustrated in FIGS. 6A and 6B, comparison of change in inflammatory cytokine levels over the supplementation period between groups revealed statistically significant reductions in IL-1β and TNF-α. A statistically significant difference was observed for change in IL-1β between placebo and active groups (p<0.001), and a very large effect size of d=1.095 (95% CI=(0.489,1.694)) was noted. Similarly, a statistically significant difference was seen for change in TNF-α between placebo and active groups (p=0.003) with a large Cohen's effect size d=0.890 (95% CI=(0.293, 1.482)). A statistically significant difference was not observed for change in IL-6 between placebo and active groups with p=0.349 and Cohen's effect size estimate d=0.277 (95% CI=(−0.302,0.855)). Finally, as illustrated in FIG. 7, a statistically significant difference was observed for change in oxidized low-density lipoprotein between placebo and active groups (p=0.009), with a large Cohen's effect size of d=0.820 (95% CI=(0.206,1.429)). The mean increase of oxidized low-density lipoprotein in the placebo group over study period was 50.37 nanograms/milliliter (ng/mL) (±159.55) while the mean decrease in the active group over study period was 90.44 nanograms/milliliter (ng/ml) (±173.96). No statistically significant differences were observed for any parameters based on supplement form or suspension (with p>0.05 for all comparisons), as confirmed by Tukey post-hoc analysis.

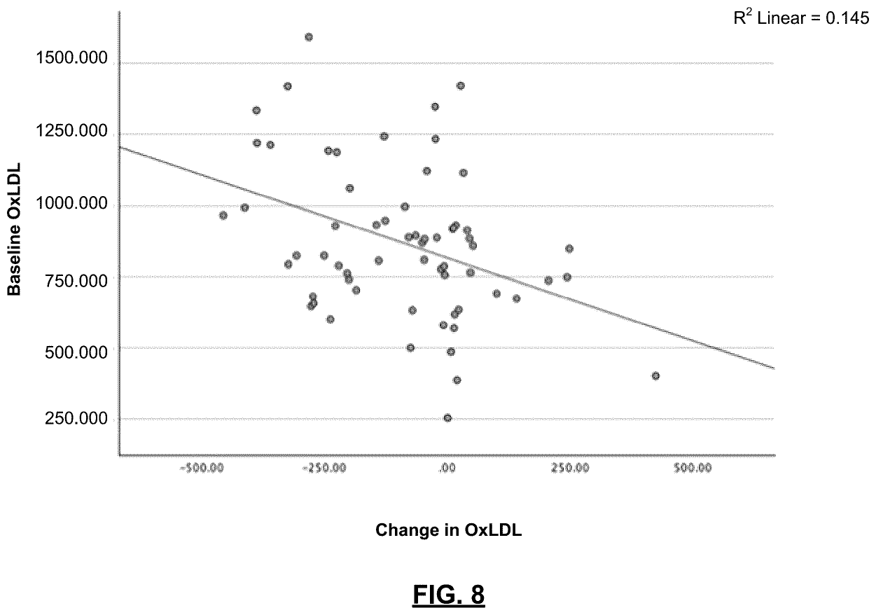

As illustrated in Table 1, and also FIGS. 5-7, with lutein, zeaxanthin, and meso-zeaxanthin supplementation, a significant decrease in oxidized low-density lipoprotein and a coincident reduction in IL-1β and TNF-α occurs within the serum, suggesting that these xanthophyll carotenoids are acting systemically to attenuate oxidative products before they may contribute to atherosclerotic plaque formation. It is worth noting that IL-6 is a ubiquitously and generously activated inflammatory cytokine, so it is not surprising that the data did not capture any direct reductions. It is likely that with the modest sample size any reduction in IL-6 would be masked by acute inflammation events (e.g., minor injury, exercise). Individual variability in inflammation status might also explain the difference observed in baseline IL-1β, although IL-1β is associated with more chronic inflammatory conditions (e.g., osteoarthritis). The most notable and applicable finding is that lutein, zeaxanthin, and meso-zeaxanthin appear to have a direct role in decreasing serum oxidized low-density lipoprotein and this presumably would act to a slow/stop the transformation of macrophage to foam cell, and therefore, slow plaque formation and decrease the impact of the cytokine-driven inflammatory feedback loop in the presence of pro-atherogenic stimuli, as illustrated, for example, in FIG. 1B. Of note, as illustrated in FIG. 8, the impact of the carotenoid intervention on oxidized low-density lipoprotein appears to be greater for participants with a higher baseline value, where a moderate inverse correlation was noted between change over time and baseline oxidized low-density lipoprotein (r=−0.341, p=0.006). Over time this reduction of inflammation and oxidation would presumably reduce the impact of a high-fat diet and poor health habits on the cardiovascular system and ultimately slow the progression toward atherosclerosis and cardiovascular disease.

As illustrated in Table 1, and also FIGS. 5-8, oil suspension (sunflower vs. omega-3s) did not show any statistically significant differences for any parameters of interest, which supports the understanding that lutein, zeaxanthin, and meso-zeaxanthin are the primarily factors influencing the observed reductions. In sum, the data shows that macular xanthophyll supplementation can decrease serum pro-inflammatory cytokines (IL-1β and TNF-α) and oxidized low-density lipoprotein and by extension may limit atherosclerotic plaque formation by disrupting the oxidation of low-density lipoprotein, which is a necessary step in foam cell formation, intimal wall deposition, and continuation of the inflammatory feedback loop. Oxidative stress is a common denominator in many pathological processes, especially those that are affected by cholesterol transfer across the endothelium within the cardiovascular system. Cardiovascular disease is globally the leading cause of death, and supplementation with dietary xanthophylls are here shown to decrease oxidation and inflammation within the cardiovascular system, which is impactful and clinically relevant for much of the population.

### Example 2

Example dietary supplements including macular xanthophylls may be prepared and administered to a human subject in accordance with various aspects of the present disclosure. For example, Table 2 compares different baseline (or starting) variables between a first group receiving the dietary supplement including macular xanthophylls in accordance with various aspects of the present disclosure and a placebo group. The active (or first) group may include sixty-six participants, while the placebo (or second) group includes fourteen participants.

TABLE 2

| | Placebo | Active | |
|---|---|---|---|
| Variable | (n = 14) | (n = 66) | Sig. |
| Age (Years) | 45.79 ± 9.37 | 44.77 ± 10.63 | 0.740 |
| Body Mass Index | 25.18 ± 3.50 | 28.18 ± 5.84 | 0.069 |
| (kg/m²) | | | |
| Smoking Risk | 5.63 ± 10.44 | 5.19 ± 8.21 | 0.860 |
| Skin Scanner | 36762 ± 8852 | 36184 ± 13721 | 0.881 |
| DHA | 178.10 ± 55.08 | 173.48 ± 59.07 | 0.789 |
| AOP | 0.898 ± 0.077 | 0.889 ± 0.062 | 0.660 |
| TNFα | 5.721 ± 3.732 | 7.478 ± 3.763 | 0.116 |
| IL-1β | 1.864 ± 1.429 | 2.845 ± 1.586 | 0.036 |
| IL-6 | 3.861 ± 2.506 | 5.061 ± 3.383 | 0.214 |
| OxLDL | 912.62 ± 323.82 | 847.28 ± 302.81 | 0.471 |

As illustrated in Table 3, after the administration of the dietary supplement including macular xanthophylls (e.g., about six-month supplementation including over 20 milligrams (mg) of mixed carotenoids and at least 1 gram (g) of fish oil including eicosapentaenoic acid (EPA) and docosahexaenoic acid (DHA)), a statistically significant different may be observed for change in IL-1β between the placebo group and the active group.

TABLE 3
Impact of Intervention on Primary Outcome Measure (POM) IL-1β

| | | | 95% Confidence Interval | |
|---|---|---|---|---|
| | SIG. | Cohen's d | Lower | Upper |
| Change IL-1β | <0.001 | 1.095 | 0.489 | 1.694 |

As illustrated in Table 4, after the administration of the dietary supplement including macular xanthophylls, a statistically significant difference may not be observed for change in IL-6 between the placebo group and the active group.

TABLE 4
Impact of Intervention on Primary Outcome Measure (POM) IL-6

95% Confidence Interval

| | SIG. | Cohen's d | Lower | Upper |
|---|---|---|---|---|
| Change IL-6 | 0.349 | 0.277 | −0.302 | 0.855 |

As illustrated in Table 5, after the administration of the dietary supplement including macular xanthophylls, a statistically significant difference may be observed for change in TNF-α between the placebo group and the active group.

TABLE 5
Impact of Intervention on Primary
Outcome Measure (POM) TNF-α

| | | | 95% Confidence Interval | |
|---|---|---|---|---|
| | SIG. | Cohen's d | Lower | Upper |
| Change TNF-α | 0.003 | 0.890 | 0.293 | 1.482 |

As illustrated in Table 6, after the administration of the dietary supplement including macular xanthophylls, a statistically significant difference may not be observed for in antioxidant potential (AOP) between the placebo group and the active group at the 5% level of significance. However, as illustrated, differences between the groups may approach statistical difference and a moderate difference across the group may be noted.

TABLE 6
Impact of Intervention on Primary
Outcome Measure (POM) Change AOP

95% Confidence Interval

| | SIG. | Cohen's d | Lower | Upper |
|---|---|---|---|---|
| Change AOP | 0.073 | −0.0551 | −1.150 | 0.052 |

As illustrated in Table 7, a statistically significant difference was observed for change in oxidative low-density lipoproteins (OxLDL) between the placebo group and the active group.

TABLE 7
Impact of Intervention on Primary Outcome Measure
(POM) Oxidative Low-Density Lipoproteins (OxLDL)

| | | | 95% Confidence Interval | |
|---|---|---|---|---|
| | SIG. | Cohen's d | Lower | Upper |
| Change OxLDL | 0.009 | 0.820 | 0.206 | 1.429 |

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for improving cardiovascular health in a human subject, the method comprising:
administering a composition to the human subject identified as being at risk for cardiovascular disease, the composition including macular xanthophylls.

2. The method of claim 1, wherein the composition is provided as a single consumable including lutein, zeaxanthin, and meso-zeaxanthin.

3. The method of claim 2, wherein the single consumable includes 10 milligrams of lutein, 2 milligrams of zeaxanthin, and 10 milligrams of meso-zeaxanthin.

4. The method of claim 1, wherein the macular xanthophyll include lutein, zeaxanthin, and meso-zeaxanthin.

5. The method of claim 4, wherein the composition is provided as two or more consumables.

6. The method of claim 4, wherein the composition is administered to the human subject at least three times a week.

7. The method of claim 4, wherein the composition is administered to the human subject at least two times a week.

8. The method of claim 4, wherein the composition is administered to the human subject daily.

9. The method of claim 1, wherein the composition further includes:
an omega-3 fatty acid.

10. The method of claim 9, wherein the omega-3 fatty acid is provided as a free acid, a salt, a triglyceride, or any combination thereof.

11. The method of claim 9, wherein the omega-3 fatty acid includes:
docosahexaenoic acid (DHA), eicosapentaenoic acid (EPA), or a combination of docosahexaenoic acid (DHA) and eicosapentaenoic acid (EPA).

12. The method of claim 1, wherein the composition further includes:
Vitamin A, Vitamin C, Vitamin D3, Vitamin E, Vitamin B1, Vitamin B2, niacin, Vitamin B6, folic acid, Vitamin B12, biotin, panthothenic acid, calcium, iron, iodine, magnesium, zinc, copper, choline, selenium, manganese, ginger, or any combination thereof.

13. A method for improving cardiovascular health in a human subject, the method comprising:
administering a composition to the human subject identified as being at risk for cardiovascular disease, the composition including macular xanthophylls and an omega-3 fatty acid, the macular xanthophylls including lutein, zeaxanthin, and meso-zeaxanthin.

14. The method of claim 13, wherein the composition is provided as a single consumable.

15. The method of claim 14, wherein the single consumable includes 10 milligrams of lutein, 2 milligrams of zeaxanthin, and 10 milligrams of meso-zeaxanthin.

16. The method of claim 13, wherein the composition is provided as two or more consumables.

17. The method of claim 13, wherein the omega-3 fatty acid includes:
docosahexaenoic acid (DHA), eicosapentaenoic acid (EPA), or a combination of docosahexaenoic acid (DHA) and eicosapentaenoic acid (EPA).

18. The method of claim 13, wherein the composition further includes:
Vitamin A, Vitamin C, Vitamin D3, Vitamin E, Vitamin B1, Vitamin B2, niacin, Vitamin B6, folic acid, Vitamin B12, biotin, panthothenic acid, calcium, iron, iodine, magnesium, zinc, copper, choline, selenium, manganese, ginger, or any combination thereof.

19. A composition for improving cardiovascular health in a human subject, the composition comprising:
lutein;
zeaxanthin;
meso-zeaxanthin macular xanthophylls; and
an omega-3 fatty acid.

20. The composition of claim 19, wherein the omega-3 fatty acid includes:
docosahexaenoic acid (DHA), eicosapentaenoic acid (EPA), or a combination of docosahexaenoic acid (DHA) and eicosapentaenoic acid (EPA).
